(19) United States
(12) Patent Application Publication
(10) Pub. No.: US 2005/0064047 A1
(43) Pub. Date: Mar. 24, 2005

(54) Composition and method for treating skin disorders

(76) Inventor: Brian Penick, Coral Springs, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 10/669,975

(22) Filed: Sep. 24, 2003

(51) Int. Cl. A61K 33/04; A61K 31/555; A61K 31/496

(52) U.S. Cl. 424/702; 514/184; 514/254.07; 424/703

(57) ABSTRACT

A composition for treating skin conditions includes sulfur combined with a carrier vehicle such as, VASELINE®, petroleum jelly, lard or the like. At least fifty % by weight of the sulfur is used in combination with the VASELINE®, petroleum jelly or lard to create an effective skin cream for curing skin conditions such as, tinea versicolor, sebhorrheic keratosis and other kinds of dermatitis. The skin cream generally requires one application, unless the condition is severe, thereby avoiding daily and multi-daily expensive regimens. Additional components such as pyrithione zinc, selenium sulfide and ketoconazole, in varying weight proportions, typically between 1% and 2.5%, and in various combinations of each, may also be combined with sulfur in the carrier vehicle to provide enhanced treatment of various skin conditions.

### CROSS-REFERENCE TO RELATED APPLICATION

This application is related to and claims priority to U.S. patent application Ser. No. 10/037,033, filed Oct. 29, 2001, entitled SUNSPOT SKIN CREAM, the entirety of which is incorporated herein by reference.

### STATEMENT REGARDING FEDERALLY SPONSORED RESEARCH OR DEVELOPMENT

n/a

### FIELD OF THE INVENTION

The present invention relates to a composition and method for treating skin conditions and more particularly to a sulfur-based composition that is effective in treating skin conditions such as but not limited to sunspots, otherwise known as tinea versicolor, foot fungus and tinea cruris, commonly known as jock itch.

### BACKGROUND OF THE INVENTION

Tinea versicolor is a very common skin condition, especially in sub tropical regions. It appears as a patch or patches of white, brown or light pink, finely flaking skin. This condition can get worse enough to cover the entire upper body including the face, sometimes causing itching and burning.

Tinea versicolor is caused by the yeast pityrosporon orbiculare. People who have tinea versicolor are genetically predisposed to developing a rash when this germ is present on the skin. When the yeast grows on untanned shin, the rash is pink to brown. When the yeast grows on tanned skin, the rash looks white because the yeast blocks out the sunlight and the skin where the yeast is growing does not tan. When growing on Asian or African-American skin, the rash can look darker or lighter than the surrounding skin depending on the patient.

Conventional treatments of tinea versicolor are commonly ineffective. Oral ketaconazole may be prescribed in doses of 200 mg daily for 2 to 4 weeks. Various treatment regimes utilizing oral ketaconazole have been reported to be effective in preventing relapse, but such regimens may not be as effective as reported. This oral prescription has many side effects and past history of liver disease and abnormal liver function test are relative contraindications. Fulminating hepatitis has been reported to be associated with oral ketaconazole and it thus should not be used to treat the skin infection tinea versicolor.

Another conventional treatment is through the use of SELSUN® Shampoo (selenium sulfide). SELSUN® Shampoo is applied to the affected areas before bed and washed off the next morning. This procedure is repeated for several days. Some treatments make use of Lamasil (terbinafine hydrochloride) as a pill or as a topical solution. As a topical solution, Lamasil must be applied several times per day for several weeks and even then may be an ineffective treatment. The use of oral medication is undesired as well, because the oral medications conventionally prescribed for these skin disorders have side effects and do not offer a permanent solution to the problem.

Needs therefore exist for a safe and efficient treatment of skin irritations such as tinea versicolor, sebhorrheic keratosis, foot fungus, and tinea cruris.

### SUMMARY OF THE INVENTION

The present invention is an effective alternative to conventional treatments of tinea versicolor and other skin disorders. The present skin cream may be used to treat skin conditions including, but not limited to, tinea versicolor and sebhorrheic keratosis. The skin cream preferably requires one application, unless the condition is severe, to cure the condition. In contrast, conventional treatments require daily and multi-daily regimens for days or months making them expensive and ineffective if a strict regimen is not followed.

The present invention uses sublimed sulfur in a greater percentage than is currently used in any conventional treatment. Conventional treatments expressly avoid excessive sulfur content and advocate minimal amounts of sulfur to be used in the creams and lotions.

In a preferred embodiment, the skin cream mixture or composition is topically applied to the irritated skin and is generally effective in treating the skin disorder after one application. The cream may generally be applied after the subject showers and before bedtime. The cream is applied to all affected areas. Upon waking, the subject showers to remove any excess residual cream.

The present invention, when applied topically, is effective in treating many different skin disorders, but in particular is significantly effective in treating tinea cruris (jock itch), tinea pedis (foot fungus), tinea versicolor (sunspots), and seborrheic keratoses.

In one embodiment of the invention, a topical composition for the treatment of skin conditions is provided comprising, sulfur, selenium sulfide, and a carrier vehicle, wherein a weight percent of the sulfur is at least equal to or greater than a weight percent of the carrier vehicle. Ketoconazole and/or pyrithione zinc may be added either in lieu of or in combination with the above composition in varying quantities. Typically, combinations of selenium sulfide, ketoconazole and pyrithione zinc are added to the sulfur-based composition in amounts varying between 1% and 2.5%, by weight. Any combination of selenium sulfide, ketoconazole and/or pyrithione zinc may be included in the composition provided both sulfur and the carrier vehicle are present and the weight percent of the sulfur is at least equal to or greater than a weight percent of the carrier vehicle. For example, the composition may include only sulfur and selenium sulfide in a carrier vehicle; sulfur, selenium sulfide and ketoconazole in a carrier vehicle; sulfur, selenium sulfide, ketoconazole and pyrithione zinc in a carrier vehicle; sulfur and ketoconazole in a carrier vehicle; sulfur, pyrithione zinc, and ketoconazole in a carrier vehicle; sulfur and pyrithione zinc in a carrier vehicle; or sulfur, selenium sulfide and pyrithione zinc in a carrier vehicle.

### DETAILED DESCRIPTION OF THE INVENTION

In a preferred embodiment the present invention includes a composition having a vehicle or carrier and sulfur. Preferably, the mixture or composition comprises approximately equal parts of the carrier vehicle and sulfur in the absence of water.

Preferably, equal parts of sublimed sulfur and the carrier vehicle, i.e. in ratios of 1:1, are used to form the cream. The carrier vehicle includes, but is not limited to, VASELINE®, petroleum jelly, or lard or any similar component or combinations thereof, with the same overall end effect. The essential component in the composition is the sulfur.

In another preferred embodiment, the sulfur is combined with the carrier, such as VASELINE®, in a ratio in which the sulfur is greater than the carrier.

The preferred skin cream is applied directly to the skin to cure skin irritations, dermatitis, and diseases such as, but not limited to, tinea versicolor (sunspots) and seborrheic keratoses. The skin cream may be in any form suitable for topical application such as, but not limited to, gels, creams, lotions, ointments, powders, pastes, sprays or the like.

A preferred method of treating skin conditions includes preparing the composition, preferably as a non-aqueous topical application composition by mixing sulfur and a carrier vehicle, and applying the topical composition to areas of interest on the skin for treating skin conditions. Preferably, equal percents by weight of the sulfur and the carrier vehicle are mixed together, such that the percent by weight of sulfur is greater than the percent by weight of the carrier vehicle.

The sulfur may be in the form of sublimed sulfur and the carrier vehicle may include, but is not limited to, VASELINE®, lard, petroleum jelly, etc. The composition may be prepared for use as a topical application and may be in any suitable form such as, but not limited to, lotions, creams, ointments, powders, gels, pastes, sprays, and combinations thereof.

The following exemplify various compositions of the inventive skin cream.

### EXAMPLE 1

| | |
|---|---|
| Sulfur | 50-100% wt. or % vol. |
| Carrier | 0 to 50% wt. or % vol. |

### EXAMPLE 2

| | |
|---|---|
| Sulfur | 50-100% wt. or % vol. |
| VASELINE ® | 0 to 50% wt. or % vol. |

### EXAMPLE 3

| | |
|---|---|
| Sulfur | 50-100% wt. |
| Petroleum Jelly | 0 to 50% wt. or % vol. |

### EXAMPLE 4

| | |
|---|---|
| Sulfur | 50-100% wt. or % vol. |
| Lard | 0 to 50% wt. or % vol. |

In addition to the sulfur base in a carrier vehicle, additional components may be added to the composition that enhances its effectiveness in treating skin disorders. In one embodiment, a topical composition for treatment of skin conditions is provided comprising, sulfur, selenium sulfide, and a carrier vehicle, wherein a weight percent of the sulfur is at least equal to or greater than a weight percent of the carrier vehicle. Selenium sulfide is derived from the chemical compound of selenium, which is a mineral essential to the human body. Selenium sulfide is effective as a topical composition to treat and control various fungal-based skin disorders. Although virtually any percentage by weight of selenium sulfide may be used in the composition, typically, between 1% and 2.5%, by weight, of selenium sulfide is effective in treating the fungal aspects of skin disorders.

Ketoconazole may be added to the composition, either with or without the presence of selenium sulfide, to provide further effectiveness in the treatment of skin disorders. Ketoconazole, when applied topically, is effective in treating various fungal infections and skin conditions. Although virtually any percentage by weight of Ketoconazole may be used in the topical composition, typically, between 1% and 2.5%, by weight of Ketoconazole is effective in treating the fungal aspects of skin disorders.

Pyrithione Zinc is another ingredient that may be added to the composition, either with or without the presence of selenium sulfide and/or Ketoconazole, to provide further effectiveness in the treatment of skin disorders. Pyrithione zinc is a zinc derivative and acts as an antibacterial and antifungal agent in the treatment of various skin conditions. Virtually any percentage by weight of Pyrithione Zinc may be used in the composition. Typically, between 1% and 2.5%, by weight of Pyrithione Zinc is effective in treating the fungal aspects of skin disorders.

While the invention has been described with reference to specific embodiments, modifications and variations of the invention may be constructed without departing from the scope of the invention, which is defined in the following claims.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A topical composition for treatment of skin conditions comprising sulfur, selenium sulfide, and a carrier vehicle wherein a weight percent of the sulfur in the topical composition is at least equal to or greater than a weight percent of the carrier vehicle.

2. The topical composition of claim 1 further comprising ketoconazole.

3. The topical composition of claim 2 further comprising pyrithione zinc.

4. A topical composition for treatment of skin conditions comprising, sulfur, ketoconazole, and a carrier vehicle, wherein a weight percent of the sulfur in the topical composition is at least equal to or greater than a weight percent of the carrier vehicle.

5. The topical composition of claim 4 further comprising pyrithione zinc.

6. A topical composition for treatment of skin conditions comprising, sulfur, pyrithione zinc, and a carrier vehicle, wherein a weight percent of the sulfur in the topical composition is at least equal to or greater than a weight percent of the carrier vehicle.

7. The topical composition of claim 6 further comprising selenium sulfide.

8. The topical composition of claim 3 wherein the composition comprises between substantially 1% and substantially 2.5% by weight of selenium sulfide, between substantially 1% and substantially 2.5% by weight of ketoconazole, and between substantially 1% and substantially 2.5% by weight of pyrithione zinc.

9. The topical composition of claim 1, wherein the sulfur is sublimed sulfur.

10. The topical composition of claim 1, wherein the carrier vehicle is VASELINE®.

11. The topical composition of claim 1, wherein the carrier vehicle is petroleum jelly.

12. The topical composition of claim 1, wherein the carrier vehicle is lard.

13. The topical composition of claim 1, wherein the carrier vehicle is a lotion.

14. The topical composition of claim 1, wherein the composition is a cream.

15. The topical composition of claim 1, wherein the composition is an ointment.

16. The topical composition of claim 1, wherein the composition is a powder.

17. The topical composition of claim 1, wherein the composition is a gel.

18. The topical composition of claim 1, wherein the composition is a paste.

19. The topical composition of claim 1, wherein the composition comprises at least about fifty percent by weight sulfur.

20. The topical composition of claim 1, wherein the composition comprises at least about sixty percent by weight sulfur.

21. The topical composition of claim 1, wherein the composition comprises at least about seventy percent by weight sulfur.

22. The topical composition of claim 1, wherein the composition comprises at least about eighty percent by weight sulfur.

23. The topical composition of claim 1, wherein the composition comprises at least ninety percent by weight sulfur.

24. The topical composition of claim 1, wherein the composition comprises about 50-100 percent by weight sulfur.

25. The topical composition of claim 1, wherein the composition is non-aqueous.

26. A method of treating skin conditions, the method comprising preparing a non-aqueous composition by mixing sulfur, selenium sulfide and a carrier vehicle together, and applying the topical composition to the skin.

27. The method of claim 26 wherein preparing the non-aqueous composition further comprises mixing ketoconazole with the sulfur, the selenium sulfide and the carrier vehicle.

28. The method of claim 27 wherein preparing the non-aqueous composition further comprises mixing pyrithione zinc with the sulfur, the selenium sulfide, the ketoconazole and the carrier vehicle.

29. The method of claim 26, wherein the mixing further comprises mixing equal percents, by weight, of the sulfur and the carrier vehicle.

30. The method of claim 26, wherein the mixing further comprises mixing a percent, by weight, of sulfur greater than a percent by weight of the carrier vehicle.

31. The method of claim 26, wherein the treating comprises treating dermatitis.

32. The method of claim 26, further comprising treating skin conditions selected from a group consisting of tinea versicolor or sunspots, seborrheic keratoses, foot fungus and tinea cruris.

33. The method of claim 26, wherein the mixing further comprises mixing sublimed sulfur with the carrier vehicle.

34. The method of claim 26, wherein the mixing further comprises selecting the carrier vehicle from a group consisting of VASELINE®, lard, petroleum jelly, and combinations thereof.
